(19) United States
(12) Patent Application Publication
(10) Pub. No.: US 2004/0063153 A1
(43) Pub. Date: Apr. 1, 2004

(54) Method for isolation of protein complexes using affinity binding

(76) Inventors: Tomas Jelinek, Lake Placid, NY (US); Arthur LaMarche, Lake Placid, NY (US); Shannon K. Boyd, Saranac Lake, NY (US); Dominic Eisinger, Keene, NY (US)

Correspondence Address: Carella, Byrne, Bain, Gilfillan, Cecchi,, Stewart & Olstein, 6 Becker Farm Road, Roseland, NJ, 07068, US

(21) Appl. No.: 10/616,725

(22) Filed: Jul. 10, 2003

Related U.S. Application Data
(60) Provisional application No. 60/402,780, filed on Aug. 12, 2002.

(51) Int. Cl. G01N 33/53

(52) U.S. Cl. 435/7.1

(57) ABSTRACT

A method for the isolation and purification of a polypeptide or protein of interest as part of an antigen-antibody complex using a spin column is disclosed, wherein such methods employ either an antibody specific ligand derived from protein A and a spin column containing a resin that binds said ligand, or a spin column containing a nickel-chelate resin capable of directly binding an antibody. Kits containing compositions and spin columns useful in the purification methods are also described.

The present application claims priority of U.S. Provisional Application Serial No. 60/402,780, filed Aug. 12, 2002, the disclosure of which is hereby incorporated by reference in its entirety.

### FIELD OF THE INVENTION

The present invention relates to the field of protein purification, especially the isolation and purification of antigen-antibody complexes, using affinity columns comprising modified fragments of protein A reversibly bound to a solid phase matrix, and with the unique advantage that the proteins are in their native configuration and thus retain enzymatic activity.

### BACKGROUND OF THE INVENTION

Immunoprecipitation is a technique designed to isolate a specific protein of interest for further uses. Non-limiting examples include assay of enzymatic activity, electrophoresis, western blotting, or identification and characterization of other associated proteins or nucleic acids or for use as vaccines and for other purposes for which isolated and purified proteins are advantageous.

Conventional immunoprecipitation is commonly carried out by incubation of a sample containing the antigen with antibody that specifically binds said antigen, and optionally immobilized affinity resin, such as protein A covalently linked to agarose or Sepharose™. The sample consists of solid and liquid phases, and must be processed with much care during several wash steps, to enrich for immune complexes. Washing must remove residual fluid as completely as possible to achieve desired purification, yet must not result in loss of solid phase beads through inaccurate aspiration. These problems combine to limit the reproducibility of such techniques.

An improvement upon this was made by Pierce (a brand of Perbio, Rockville, Ill., product numbers 45213, 45216, 45217, 45218, 45219)), offering a system by which the above mixture, with or without additional modifications, is introduced into a polypropylene tube with a filter bottom, colloquially called a basket, allowing washes to occur by centrifugation and constant flow of fluid within the basket through the sample. However, while this procedure allows one the option to covalently link antibody to protein-A agarose, place it into a basket with porous bottom, and conduct immunoprecipitation, it does not facilitate ready elution of the desired product.

With both above techniques, the immune complex must be eluted from the solid phase by one of several harsh techniques, including acid exposure (low pH), or by denaturing buffers containing strong detergents, often with concomitant heating. However, for the study of cellular enzymes, these elution conditions are unsuitable for recovery of active enzyme, leaving as the only remaining option the conducting of enzyme assays on immobilized material, which significantly limits the flexibility of those assays.

The present invention solves these problems by providing a means for isolation of immune complexes in spin-column format, but advantageously allows elution of the immune complexes with gentle buffer conditions so the complex retains native enzymatic activity. In accordance with this invention, it has been found that certain antibodies bind directly to nickel-chelate resins, and can be eluted by agents that compete with histidine residues for nickel-binding, such as imidazole.

In one report, the authors tagged intact protein A with the above groups, and showed that immune complexes could be bound and eluted (See Poon, R Y, and Hunt, T., Reversible immunoprecipitation using histidine- or glutathione S-transferase-tagged staphylococcal protein A. Anal Biochem 218:26-33 (1994). Conversely, the present invention utilizes a single domain of the binding protein, and we use centrifuge spin-columns to make the process faster and more reliable.

### BRIEF SUMMARY OF THE INVENTION

In one aspect, the present invention relates to a method for isolating a polypeptide as a purified polypeptide-antibody complex from a sample containing the polypeptide comprising:

(a) contacting an antibody capture affinity ligand (ACAL) with a sample containing a polypeptide to be recovered from said sample, in the presence of an antibody that binds to said polypeptide to be isolated, under conditions supporting contacting, to form a complex of said polypeptide, antibody and ACAL;

(b) introducing the complex formed in step (a) to a spin column comprising a resin that binds said ACAL;

(c) centrifuging the column of (b);

(d) optionally washing the column of (c) with lysis buffer; and

(e) washing the column from (d) with elution buffer to elute the polypeptide-antibody complex;

thereby recovering a purified polypeptide-antibody complex.

In another aspect, the present invention relates to a method for isolating a polypeptide as a purified polypeptide-antibody complex from a sample containing the polypeptide comprising:

(a) contacting said sample, containing a polypeptide to be recovered, with an antibody that binds said polypeptide, under conditions promoting said contacting, to form a polypeptide-antibody complex;

(b) introducing the complex formed in step (a) to a spin column containing a nickel-chelate resin that binds said antibody;

(c) centrifuging the column of (b);

(d) optionally washing the column of (c) with lysis buffer; and

(e) washing the column from (d) with elution buffer to elute the polypeptide-antibody complex;

thereby recovering a purified polypeptide-antibody complex.

In a further aspect, the present invention relates to a kit comprising a set of instructions for carrying out the method, or methods, of the invention and a spin column.

### BRIEF DESCRIPTION OF THE DRAWINGS

FIG. 1 shows a schematic flowchart of one embodiment of the invention for antigen-antibody complex isolation.

FIG. 2 shows an example of immunoprecipitation of cdk2 from HeLa cells utilizing the method of the invention as described in detail in Example 1. Here, the lanes are as follows:

Lanes 1-4: Rabbit IgG

Lanes 5-9: Anti-cdk2

Lanes 1, 5: Starting material removed at start of IP, used as standard for quantity loaded.

Lanes 2, 6: Flowthrough (material that did not stick in the column).

Lanes 3, 7: Eluted material

Lanes 4, 8: Elution repeated for any residual material coming off the column.

### DETAILED DESCRIPTION OF THE INVENTION

The present invention contemplates the use of a spin column in the isolation and preparation of polypeptides or proteins of interest and utilizing an antibody that reacts with, binds to, or is otherwise specific for, the polypeptide or protein to be isolated and a ligand that binds the antibody to a resin contained in the spin column.

As used herein, the term “spin-column” means a modified microcentrifuge tube with a spout in the bottom, and with a layer of material designed to specifically bind material of interest. Its function is based on centrifugal flow of liquid from an enclosed upper chamber above said layer, through the layer, and into a capture tube. It should be noted that there is no reason for such layer to be immobilized in that it is a feature of the present invention that removing the barrier above the resin, thereby allowing at least some mixing with the solution greatly improves the results. Longer mixing time is advantageous.

Protein A (PrA) is a component of the cell wall of Staphylococcus aureus and is a specific immunoglobulin-binding protein. The protein consists of a C-terminal cell wall anchoring region and 5 highly homologous independent binding domains, each with individual molecular weights of 6.6 kDa that are connected by short protease sensitive loops. Binding occurs at the Fc portion of IgG by induced fit and variable affinity exists for IgGs from different species and subclasses. Immobilized protein A, whether native or recombinant, is susceptible to protease cleavage of inter-domain regions, which can cause leaching problems. Single IgG binding domains posses more uniform IgG binding characteristics than the parent molecule and lack the protease labile inter-domain sequences.

Protein G is from group C and G streptococci. It comprises a single polypeptide with multiple binding domains linked in a cylindrical conformation. Wild-type protein G (see GenBank No. P19909) contains albumin-binding domains as well as IgG-Fc and Fab-binding domains. Protein G binds the same region of IgG-Fc as protein A. The two proteins share no homology, but have a gross conformational similarity. For use in the methods of the invention, a single Ig-binding domain was prepared encompassing residues 373-428 of protein G, and incorporating a 6-His tag also.

In accordance with the foregoing, the present invention relates to a method for isolating a polypeptide as a purified polypeptide-antibody complex from a sample containing the polypeptide comprising:

(a) contacting an antibody capture affinity ligand (ACAL) with a sample containing a polypeptide to be recovered from said sample, in the presence of an antibody that binds to said polypeptide to be isolated, under conditions promoting said contacting, to form a complex of said polypeptide, antibody and ACAL;

(b) introducing the complex formed in step (a) to a spin column comprising a resin that binds said ACAL;

(c) centrifuging the column of (b);

(d) optionally washing the column of (c) with lysis buffer; and

(e) washing the column from (d) with elution buffer to elute the polypeptide-antibody complex;

thereby recovering a purified polypeptide-antibody complex.

The sample containing the polypeptide to be complexed and isolated may be from any source, including directly from a cell lysate. In preferred embodiments, such as where the ligand comprises a histidine tag, the elution buffer of step (e) may be imidazole. In one embodiment, the lysis buffer of step (d) is a Tris based buffer.

In a preferred embodiment, the ACAL comprises a single antibody binding domain of Protein A or of Protein G, or a mixture or cocktail of these, with an attached tag on each that operates to reversibly bind the resin of step (b). In other preferred embodiments, the tag is a polyhistidine tag or wherein said tag comprises a cysteine residue at the terminus of the Protein A portion, or Protein G portion, of said ACAL, which cysteine tag produces oxidative attachment to the resin of step (b). Preferred embodiments of such method may utilize an antibody binding domain of Protein A and/or Protein G, separately or as a mixture or cocktail. Where a mixture is used, each may have separate tags, which may or may not be different from each other and may or may not permit discrimination thereof. While these proteins may not naturally have a C-terminal cysteine, this residue is readily added to the protein (a modification that worked well with protein A).

To facilitate the methods disclosed herein, a synthetic mini-gene encoding the PrA B-domain was prepared by assembling 4 overlapping oligonucleotides by standard procedures (3). Several useful design features were incorporated into the mini-gene. The preferred codon usage for E. coli was used to help ensure maximal protein expression, and a C-terminal six histidine tag was added for purifying the protein by Immobilized Metal Affinity Chromatography (IMAC) (3). Our synthetic B-domain is efficiently expressed as a soluble protein that migrates close to its predicted molecular weight of 9.2 kDa. Also, the active protein is easily isolated in high yield and purity after a single IMAC purification step.

PrA has been used for purifying antibodies for over 25 years (35;40) and the B-domain peptide sequence was determined before (50) molecular cloning of the gene (12;53). The synthetic mini-gene encoding the B-domain was constructed based on that information alone although the recombinant protein made from the cloned Staphylococcus aureus gene is known (13).

Experiments confirmed that the synthetic B-domain protein binds specifically to antibodies by non-antigen dependent affinity blotting. Here, “non-antigen dependent” means that binding to the immobilized B-domain occurred via the Fc portion of the antibody and not the antigen binding domain.

A nickel-chelate affinity resin (obtained from Affiland, Ans-Liege, Belgium) was used with a pentadentate chelation configuration. This product has been used successfully with other chelators, including Ni-NTA (Qiagen, Valencia, Calif.) and trivalent nickel (obtained from Clontech Laboratories, now part of BD Biosciences, Palo Alto, Calif.)

This resin was packed into a spin-column (the Qiagen version is commercially available and has been used successfully) and used in conjunction with the protein A fragment described above to conduct immunoprecipitations.

In accordance with the method of the invention, antibody is added to a sample that contains antigen, most typically a lysate of cells. The protein A fragment is added to the mixture, which is loaded onto the spin-column, and passed through by centrifugation. Several volumes of wash buffer as passed through, and the immune complex and protein A are eluted with imidazole buffer, that competes with the 6-Histidine tag. Imidazole is a gentle buffer, and immune complexes eluted thus can be used directly for assays of the enzyme activity of the immunoprecipitated antigen.

Alternatively, the protein A fragment can be attached oxidatively to a matrix through a cysteine on the very terminus of the protein, used in immunoprecipitation, and eluted by reduction of the cysteine.

Further, it is known that some antibodies can bind directly to nickel-chelate resins and we are aware that this occurs in our system, to varying degrees with different immunoglobulin species but such has not heretofore been exploited for the purpose of reversible immunoprecipitation. The system of the present invention combines these two modalities of binding antibody-antigen complexes (through protein A fragment, and directly to the resin), with the end-result being the same—reversible immunoprecipitation in spin-column format.

In accordance with the foregoing, the present invention relates to a method for isolating a polypeptide as a purified polypeptide-antibody complex from a sample containing the polypeptide comprising:

(a) contacting said sample, containing a polypeptide to be recovered, with an antibody that binds said polypeptide, under conditions promoting said contacting, to form a polypeptide-antibody complex;

(b) introducing the complex formed in step (a) to a spin column containing a nickel-chelate resin that binds said antibody;

(c) centrifuging the column of (b);

(d) optionally washing the column of (c) with lysis buffer; and

(e) washing the column from (d) with elution buffer to elute the polypeptide-antibody complex;

thereby recovering a purified polypeptide-antibody complex.

The sample useful in this method may be any source, preferably a cell lysate.

Cell lysates can be very heterogeneous, depending on the cell line or tissue of origin, the lysis buffer and conditions, the age of the lysate, whether the lysate is fresh or frozen, and probably other variables and thus this example uses optimized conditions for the particular experiment.

In particular, the following factors may have to be resolved before utilizing the methods of the invention, although such considerations are deemed routine in applying any methodology to a new and different application or sample and are well within the routine skill of those in the art.

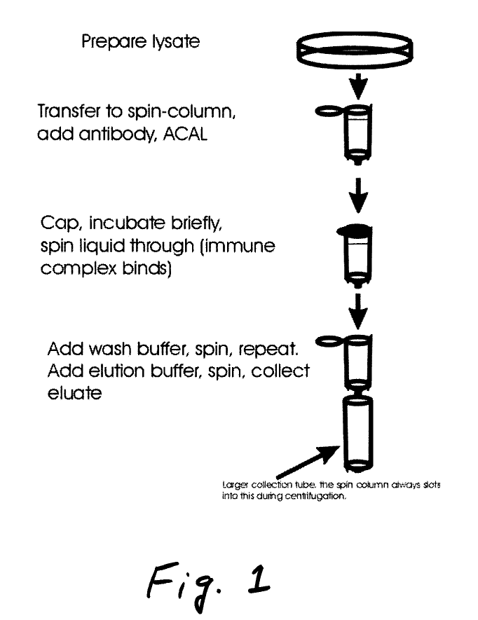

One embodiment of the present invention, using the “catch and release” procedure illustrated schematically in FIG. 1, is described below. It should be noted that all references to catalog numbers in this embodiment indicate numbers from the catalog available from Upstate Biotechnology, Inc., Lake Placid, N.Y. or on the internet at www.upstate.com. Technical notes contained in said catalog were found useful but will not be described in detail, although these are considered incorporated herein by reference. This procedure is as follows:

1. Dilute the 10× Catch and Release™ Lysis/Wash Buffer (Catalog # 20-210) to 1× using MilliQ or ultrapure water. The 1× lysis/wash buffer is used as a dilution buffer for cell lysates, to dilute the lysate to 1 mg/ml. It is also used as the wash buffer in steps 11 and 15.

2. Dilute cell lysate to 1 mg/ml using 1× Catch and Release™ Lysis/Wash Buffer from Step 1.

3. Aliquot 500 μl of diluted cell lysate (500 μg) into a blue Catch and Release™ Spin Column (Catalog # 16-195).

4. Add 4 μg of antibody (usually 2-10 μl, depending on concentration).

5. Add 10 μl (1 μg) of Antibody Capture Affinity Ligand (Catalog # 20-216).

6. Cap tube, vortex briefly, and place on a rocking platform for 15 minutes at room temperature. Here, it should be noted that standard immunoprecipitation incubations range from 1 hour to overnight at 4° C. However, this kit has been optimized for a 15 minute incubation time. We have observed no additive effect by increasing the incubation time.

7. Insert the column into a capture tube, and the capture tube in microfuge. Pulse-spin in microfuge at maximum speed for 1 minute, or until all liquid has passed through the column.

8. Wash the spin column 3× with 500 μl of 1× Catch and Release™ Lysis/Wash Buffer for 3 minutes at 2,000×g (5,000 rpm). Empty the capture tube after each spin.

9. Dilute 4× IP Elution Buffer (Catalog # 20-209) to 1× using MilliQ water and add 60 μl of diluted buffer to spin column. Here, to increase final concentration, as little as 30 μl diluted buffer may be used for elution). Insert spin column into a labeled fresh eppendorf tube in the microfuge.

10. Centrifuge at 500×g (2,500 rpm) for 2 minutes.

At this point, one can proceed to the next step for SDS-PAGE loading, or proceed to Step 12 for kinase assays

SDS-PAGE Loading:

11. Add an equal volume (30-60 μl) of 2× Reducing Sample Buffer* to eluate. Boil for 5 minutes immediately prior to loading gel. Load 20 μl per lane.

*2× Reducing Sample Buffer: 0.1M Tris HCl, pH 6.8; 3% SDS; 1% glycerol; 2.5% a-mercaptoethanol; 0.005% bromophenol blue.

In the present application, for kinase assay:

12. After Step 10 (page three), wash the spin column twice with 500 μl of 1× Catch and Release™ Lysis/Wash Buffer for 3 minutes at 2,000×g (5,000 rpm). Empty the capture tube after each spin.

13. Wash once with 500 μl of assay dilution buffer (formulation varies, depending on specific kinase assay) for 3 minutes at 2,000×g (5,000 rpm).

14. Dilute 4×IP Elution Buffer (Catalog # 20-209) to 1× using MilliQ water and add 60 μl of diluted buffer to spin column. Here, to increase final concentration, as little as 30 μl diluted buffer may be used for elution). Insert spin column into a labeled fresh eppendorf tube in the microfuge.

15. Centrifuge at 500×g (2,500 rpm) for 2 minutes.

16. Add 30-60 μl of MilliQ water or assay dilution buffer (i.e., the same volume of 1×IP Elution Buffer used in Step 14) to the eluate before proceeding with a kinase assay.

In performing such a kinase assay, it should be noted that if 2×IP Elution Buffer is used for elution prior to a kinase assay, one should dilute with 180 μl of MilliQ water or assay dilution buffer to ensure IP Elution Buffer does not interfere with kinase activity.

It should also be noted that:

a) In some cases, it may be possible to optimize elution yield by using 2×IP Elution Buffer rather than 1×IP Elution Buffer. If this procedure is used, it is recommended first following the above procedure and increasing the Elution Buffer concentration only if the eluted immunocomplex yield is unsatisfactory.

b) If the column is used for an additional elution step then the immunocomplex recovered will be more dilute than in the primary elution step.

In a further aspect, the present invention relates to a kit comprising a set of instructions for carrying out the method, or methods, of the invention and a spin column. In a further embodiment of such a kit, there may also be included one or more of antibody capture affinity ligand (ACAL), an antibody, a sample of a lysis buffer, and a sample of an elution buffer, each said member, when included in said kit, being in sufficient quantity to be useful for the isolation of at least one polypeptide by any of the methods of the invention.

In particular embodiments of such a kit, the specific components may be selected from any of the following:

1. Antibody Capture Affinity Ligand. One or more vials containing a measured amount (for example, about 5 μg) Antibody Capture Affinity Ligand in a usable amount of buffer (for example, 50 μl PBS (phosphate buffered saline). A preferred such buffer is available from Upstate Biotechnology, Inc., Lake Placid, N.Y. (Catalog # 20-216).

2. Catch and Release Lysis Buffer. One specific embodiment of such a buffer would include one vial containing 1 ml of 0.5M Tris-HCl, pH 7.4, 1.5M NaCl, 2.5% deoxycholic acid, 10% NP-40, 10 mM EDTA. This is liquid at room temperature. A preferred such buffer is available from Upstate Biotechnology, Inc., Lake Placid, N.Y. (Catalog # 20-188a).

3. 10×IP Elution Buffer. A preferred embodiment of such a component would include one vial containing 1.5 ml of 2×PBS-based IP Elution Buffer. Liquid at 4° C. A preferred such buffer is available from Upstate Biotechnology, Inc., Lake Placid, N.Y. (Catalog # 20-209).

4. Spin Columns. These can include 1, 2, 5 or more columns containing IP capture resin.

5. Reservoir tubes: variable in number—usually around the same as the number of spin columns supplied with the kit.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The invention is described in more detail in the following non-limiting example. It is to be understood that these methods and examples in no way limit the invention to the embodiments described herein and that other embodiments and uses will no doubt suggest themselves to those skilled in the art.

### EXAMPLE 1

### Isolation of cdk2 Complexes from H La C lls

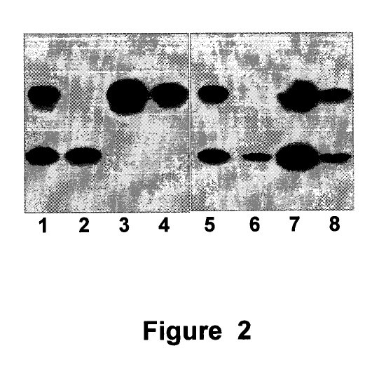

4 μg of normal rabbit IgG (obtained from Upstate Biotechnology, Inc., Lake Placid, N.Y., Catalog # 12-370) (lanes 1-4 of FIG. 2) or anti-cdk2 (obtained from Upstate Biotechnology, Inc., Lake Placid, N.Y., Catalog # 06-505) (lanes 5-8 of FIG. 2) and 1 μg of Antibody Capture Affinity Ligand were added to 500 μg of a HeLa nuclear extract, rotated for 10 minutes at room temperature and spun through the Catch and Release Spin Column for 1 minute at 15,000-RPM. The columns were washed 3× with RIPA Lysis Buffer and eluted with 60 μl of 1×IP Elution Buffer. 60 μl of Reducing Sample Buffer was added to each eluate, and 20 μl was resolved by electrophoresis, transferred to nitrocellulose and probed with anti-cdk2 (2 μg/ml). Proteins were visualized using a goat anti-rabbit secondary antibody conjugated to HRP and a chemiluminescence detection system.

The results are shown in FIG. 2 where the upper band is heavy chain of immunoglobulin, the lower band is cdk2. Very little immunoglobulin flowed through the column in either case, but cdk2 did completely when negative control was used (lane 2), and a little when the correct antibody was used (lane 6), representing that population that did not bind its antibody. No cdk2 was eluted from the negative control (lanes 3,4), but a highly enriched elution was achieved with anti-cdk2 (lane 7), and a little more on a second elution (lane 8).

What is claimed is:

1. A method for isolating a polypeptide as a purified polypeptide-antibody complex from a sample containing the polypeptide comprising:
(a) contacting an antibody capture affinity ligand (ACAL) with a sample containing a polypeptide to be recovered from said sample, in the presence of an antibody that binds to said polypeptide to be isolated, under conditions promoting said contacting, to form a complex of said polypeptide, antibody and ACAL;
(b) introducing the complex formed in step (a) to a spin column comprising a resin that binds said ACAL;
(c) centrifuging the column of (b);
(d) optionally washing the column of (c) with lysis buffer; and
(e) washing the column from (d) with elution buffer to elute the polypeptide-antibody complex;
thereby recovering a purified polypeptide-antibody complex.

2. The method of claim 1 wherein said sample is a cell lysate.

3. The method of claim 1 wherein the elution buffer of step (e) is imidazole.

4. The method of claim 1 wherein the lysis buffer of step (d) is a Tris buffer.

5. The method of claim 1 wherein said ACAL comprises a single antibody binding domain of Protein A or Protein G or mixture of the two with an attached tag that operates to reversibly bind the resin of step (b).

6. The method of claim 5 wherein said ACAL comprises a single antibody binding domain of Protein A.

7. The method of claim 5 wherein said ACAL comprises a single antibody binding domain of Protein G.

8. The method of claim 5 wherein said ACAL comprises a mixture of single antibody binding domains of Protein A and Protein G.

9. The method of claim 5 wherein said tag is a polyhistidine tag.

10. The method of claim 8 wherein the tags on the single antibody binding domain of Protein A and Protein G are detectibly different.

11. The method of claim 5 wherein said tag comprises a cysteine residue at the terminus of the Protein A or Protein G portion of said ACAL, which cysteine tag produces oxidative attachment to the resin of step (b).

12. A method for isolating a polypeptide as a purified polypeptide-antibody complex from a sample containing the polypeptide comprising:
(a) contacting said sample, containing a polypeptide to be recovered, with an antibody that binds said polypeptide, under conditions promoting said contacting, to form a polypeptide-antibody complex;
(b) introducing the complex formed in step (a) to a spin column containing a nickel-chelate resin that binds said antibody;
(c) centrifuging the column of (b);
(d) optionally washing the column of (c) with lysis buffer; and
(e) washing the column from (d) with elution buffer to elute the polypeptide-antibody complex;
thereby recovering a purified polypeptide-antibody complex.

13. The method of claim 12 wherein said sample is a cell lysate.

14. A kit comprising a set of instructions for carrying out the method of claim 1 and a spin column.

15. The kit of claim 14 further comprising at least one member selected from the group consisting of an antibody capture affinity ligand (ACAL), an antibody, a sample of a lysis buffer, and a sample of an elution buffer, each said member, when included in said kit, being in sufficient quantity to be useful for the isolation of at least one polypeptide by the method of claim 1.
